(19) United States
(12) Patent Application Publication
(10) Pub. No.: US 2014/0200419 A1
(43) Pub. Date: Jul. 17, 2014

(54) OPTICAL DETECTION OF CAROTENOID-RELATED COMPOUNDS IN HUMAN BONE AND SURROUNDING TISSUES

(71) Applicants: Igor V. Ermakov, Salt Lake City, UT (US); Thomas D. Rosenberg, Holladay, UT (US); Werner Gellermann, Salt Lake City, UT (US)

(72) Inventors: Igor V. Ermakov, Salt Lake City, UT (US); Thomas D. Rosenberg, Holladay, UT (US); Werner Gellermann, Salt Lake City, UT (US)

(21) Appl. No.: 13/744,260

(22) Filed: Jan. 17, 2013

(51) Int. Cl. A61B 5/00

(52) U.S. Cl. CPC A61B 5/0059; A61B 5/4504; A61B 5/443
USPC 600/306; 600/476

(57) ABSTRACT

The present invention is directed to measuring the levels of carotenoids and other similar chemical compounds that are present in varying degrees in human bone and surrounding tissues. The invention permits non-contact, quantitative measurements of carotenoid levels of tissues, exposed during surgery, from a safe distance. Light reflected from an exposed bone or surrounding tissue is captured and processed to accurately quantify the carotenoid content of the bone or tissue.

### CROSS-REFERENCE TO RELATED APPLICATIONS

Not Applicable.

### BACKGROUND

Carotenoids are important plant pigments routinely ingested on a daily basis via fruit and vegetable consumption. The most prevalent carotenoids consumed in North American diets include alpha-carotene, beta-carotene, lycopene, lutein, zeaxanthin and beta-cryptoxanthin.

Carotenoids can be measured in blood, in skin, in the macular region of the human retina, and in other tissues. Blood and skin carotenoid levels correlate with dietary intake of fruits and vegetables. Therefore, measurements of blood and skin carotenoid levels can serve as an objective biomarker of fruit and vegetable intake.

Fruit and vegetable consumption is generally regarded as an important factor for increased energy and overall good health. For example, high dietary consumption of fruits and vegetables has been associated with protection against a number of diseases, including various cancers, cardiovascular disease, type II diabetes, age-related macular degeneration, and pre-mature skin aging. Furthermore, carotenoids themselves have been speculated to be one of the anti-carcinogenic phyto-chemicals of plant foods and are thought to protect the tissue cells via optical filtering and/or antioxidant action.

The standard method for the measurement of carotenoids is based on biochemical high-performance liquid chromatography (HPLC) techniques. However, these HPLC techniques are highly invasive. They require that relatively large amounts of tissue be removed from the subject for subsequent tissue processing and analysis, which besides being painful, costly and inconvenient, also takes at least several hours to complete. In the course of these types of analyses, the tissue is damaged, if not completely destroyed.

Alternatively, carotenoid concentrations can be indirectly estimated via HPLC analysis of plasma or serum. Key disadvantages again are discomfort, cost and necessity of venipuncture, which may cause participation bias since subjects may be reluctant to give blood. Furthermore, carotenoid concentrations in blood fluctuate in response to recent dietary intake, with an estimated half-life of less than 12 days for beta-carotene. The situation is even worse in the human retina, where only two of the approximately ten carotenoid species circulating in blood, i.e. lutein and zeaxanthin, are taken up and are concentrated in this tissue. Consequently, there is at best poor correlation with plasma levels for this particular tissue. In general it is necessary to develop novel, non-invasive, methods for the detection of carotenoid levels directly in the tissue of interest.

Carotenoids are n-electron conjugated carbon-chain molecules and are similar to polyenes with regard to their structure and optical properties. Distinguishing features are the number, n, of the conjugated carbon double bonds (C═C bonds), the number of attached methyl side groups, and the presence and structure of attached end groups.

A noninvasive method for the measurement of carotenoids and related chemical substances in biological tissue by resonance Raman spectroscopy (RRS) is disclosed in U.S. Pat. No. 6,205,354. This technique provides for a rapid, accurate and safe determination of skin carotenoid levels that in turn can provide diagnostic information regarding fruit and vegetable consumption, nutritional supplement uptake, or can be a marker for conditions where carotenoids or other antioxidant compounds may provide disease-related diagnostic information.

In this technique, a laser or other spectrally narrow light is directed upon the tissue area of interest, such as the palm of the hand. A small fraction of the scattered light is scattered inelastically, producing the carotenoid Raman signal that is at a different frequency or corresponding wavelength than the incident laser light, and the Raman signal is collected, filtered and measured. The Raman signal can be analyzed such that the background fluorescence signal is subtracted and the result displayed and compared with known calibration standards.

A further non-invasive optical method for the assessment of skin carotenoid levels is based on reflection spectroscopy, RS. Particularly useful is a pressure-mediated version of RS that allows one to assess skin carotenoid levels after temporal removal of interfering blood chromophores. This method is disclosed in U.S. Pat. No. 8,260,402. This RS method holds promise as a particularly simple and inexpensive method since it does not require any narrow-band light sources for excitation, since it has significantly higher signal levels and therefore requires less complex instrumentation.

Basic RS has been used previously for the quantification of carotenoids in the macular region of the human retina (“macular pigment”) and in skin. In retinal reflection spectroscopy, the macular carotenoids (which in contrast to skin comprise only two carotenoid species, i.e. lutein and zeaxanthin), are derived from a double-path propagation of white light through all ocular layers from the cornea to the reflective sclera behind the retina, and back. The quantification of carotenoids is possible with the help of a multi-layer, sequential, straight-light-path transmission model, in which the individual absorption and/or scattering effects of all ocular layers are described with respective absorption and/or scattering coefficients. The retinal carotenoid levels, concentrated in the macula, are derived from a multi-parameter fit of the calculated reflection spectra to the measured spectra.

In human skin, the much stronger light scattering caused by the outer stratum corneum layer does not permit the assumption of tissue light propagation in and modeling of straight light paths. Furthermore, there is no effective internal interface that could be used as a reflector. Instead, it has been attempted to calculate carotenoid levels from first principles, taking into account the inhomogeneity of chromophore distributions in the living tissue in this earlier approach, and using a complex spectral de-convolution algorithm with multi-compartment modeling for skin chromophores.

### BRIEF SUMMARY

The present invention extends to a method and apparatus for measuring the levels of carotenoids and other nutritionally derived chemical compounds that are present in varying degrees in human bone and surrounding tissues. Specifically, the method and apparatus permit non-contact, quantitative measurements of carotenoid levels of tissues, exposed during surgery, from a safe distance that does not compromise sterile technique. The invention employs the fact that the presence of carotenoids in cancellous bone and other surrounding tissues, including fat tissue, affects the amount of light that is reflected by the bone and the surrounding tissues. Accordingly, the invention provides an apparatus and method for detecting light reflected by exposed cancellous bone or tissue and other surrounding tissues, including fat tissue, processing the reflected light to accurately quantify the carotenoid content of the bone and other tissue.

In one embodiment, the invention comprises an apparatus for optically detecting carotenoid concentrations in a trabecular human bone while the trabecular bone is exposed. The apparatus includes a light source for emitting light onto an exposed trabecular human bone, a module, a camera, and a computer system. The module receives a portion of the light that is reflected from the bone. The camera captures an image of a first band of the reflected light and an image of a second band of the reflected light. The computer system then compares the images to determine a difference between the intensities in the first and the second bands, and generates a value, based on the difference, that represents a carotenoid concentration in the exposed human bone.

In another embodiment, the invention implements a method for optically detecting carotenoid concentrations in a trabecular human bone while the bone is exposed. Light that is reflected from an exposed trabecular human bone of a first patient is received. A first band of the reflected light is filtered. A second band of the reflected light is also filtered. An image of each of the first and the second bands is captured. A difference between the intensities in the first and the second bands is determined Then, a value based on the determined difference is output. The value represents a carotenoid concentration in the exposed human bone.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an apparatus that can be used to optically detect the level of carotenoids in a patient's exposed bone or surrounding tissue during surgery;

FIG. 2A and 2B illustrate an exemplary configuration of a multispectral imaging module;

FIG. 3 illustrates graphs of the apparent absorbance of bone, fat, and cartilage for wavelengths generally in the visible spectrum;

FIG. 4 illustrates a graph that indicates the carotenoid detection band and the reference band as used by the present invention;

FIG. 5 illustrates representative samples that can be obtained using the techniques of the present invention;

FIG. 6 illustrates images of a white standard and spongy bone sample taken using the apparatus of FIG. 2 as well as a spatially resolved plot representing the difference between the images; and

FIG. 7 illustrates histograms for HPLC-derived carotenoid concentrations in bone and skin samples.

### DETAILED DESCRIPTION

The present invention extends to a method and apparatus for measuring the levels of carotenoids and other nutritionally derived chemical compounds that are present in varying degrees in human bone and surrounding tissues. Specifically, the method and apparatus permit non-contact, quantitative measurements of carotenoid levels of tissues, exposed during surgery, from a safe distance that does not compromise sterile technique. The invention employs the fact that the presence of carotenoids in cancellous bone and other surrounding tissues, including fat tissue, affects the amount of light that is reflected by the bone and the surrounding tissues. Accordingly, the invention provides an apparatus and method for detecting light reflected by exposed cancellous bone or tissue and other surrounding tissues, including fat tissue, processing the reflected light to accurately quantify the carotenoid content of the bone and other tissue.

In one embodiment, the invention comprises an apparatus for optically detecting carotenoid concentrations in a trabecular human bone while the trabecular bone is exposed. The apparatus includes a light source for emitting light onto an exposed trabecular human bone, a module, a camera, and a computer system. The module receives a portion of the light that is reflected from the bone. The camera captures an image of a first band of the reflected light and an image of a second band of the reflected light. The computer system then compares the images to determine a difference between the intensities in the first and the second bands, and generates a value, based on the difference, that represents a carotenoid concentration in the exposed human bone.

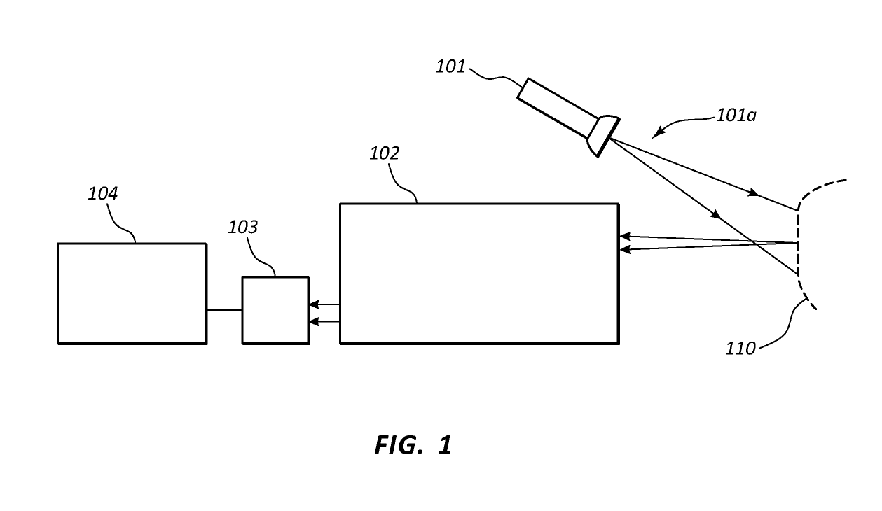

FIG. 1 illustrates an apparatus 100 that can be used to optically detect the level of carotenoids in a patient's exposed bone or surrounding tissue during surgery. Apparatus 100 includes a light source 101 for emitting light 101a onto exposed bone 110. Light source 101 can be any source of light capable of emitting light in the visible light range. A portion of light 101a is reflected by exposed bone 110 and enters multispectral imaging module 102. Multispectral imaging module 102 includes components for filtering two separate bands of the reflected light. The two bands are output to camera 103 which captures the intensity of the light within the two bands. Finally, computer system 104 analyzes the captured intensities to determine a level of carotenoids in the exposed bone 110.

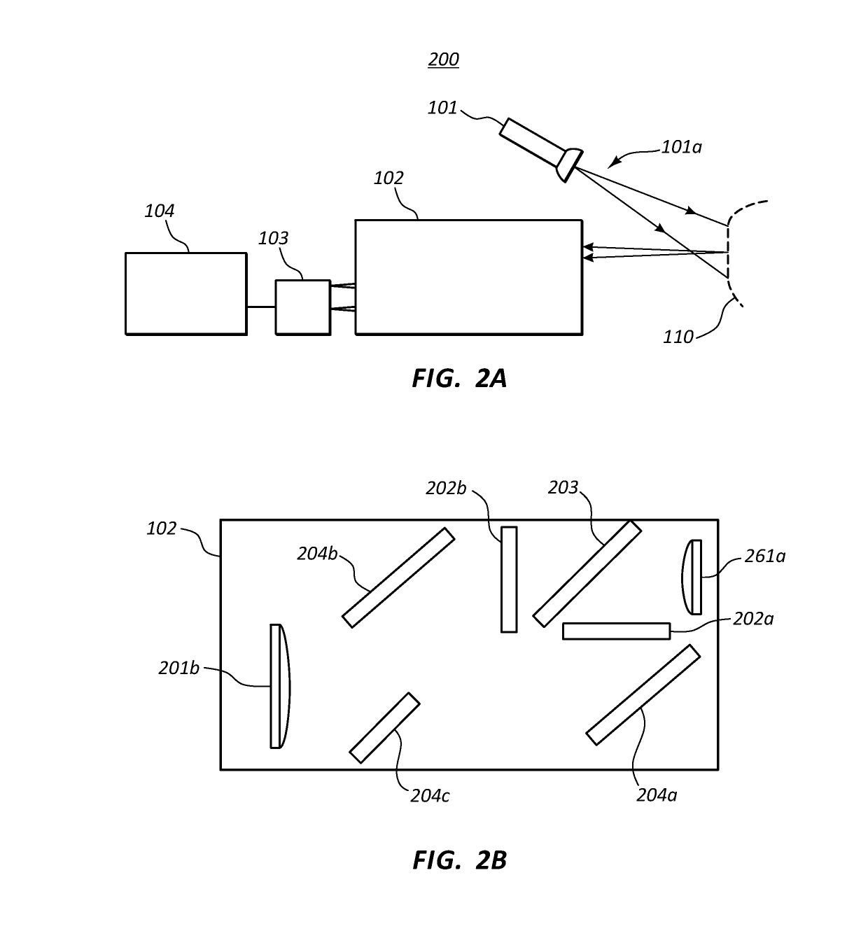

FIGS. 2A and 2B illustrate an exemplary configuration 200 of multispectral imaging module 102. As shown, multispectral imaging module 102 can include various components including lenses 201, filters 202, splitter 203, and mirrors 204. The components shown in FIGS. 2A and 2B are exemplary and any configuration of module 102 capable of functioning as a multiple band bandpass filter could be used.

Light 101a reflected from exposed bone 110 passes through lens 201a which routes the light to splitter 203. Splitter 203 splits the reflected light along two paths. One path passes through filter 202a while the other passes through filter 202b. Filters 202a and 202b each filter a different band within the visible light range.

Specifically, as further described below, the concentration of carotenoids in a bone affect the reflectance of the bone at certain wavelengths. Filters 202a and 202b, therefore, can be used to isolate a band of wavelengths that are highly affected by the presence of carotenoids in the bone as well as a band of wavelengths that are relatively unaffected by the presence of carotenoids. These two separate bands can be routed to camera 103 using mirrors 204a-204c as shown. Camera 103 captures an image of each of these bands.

Accordingly, by passing reflected light 101a through multispectral imaging module 102, two separate images can be obtained—one for each of the bands. Computer system 104 receives and processes the images to calculate a concentration of carotenoids in exposed bone 110 as further described below.

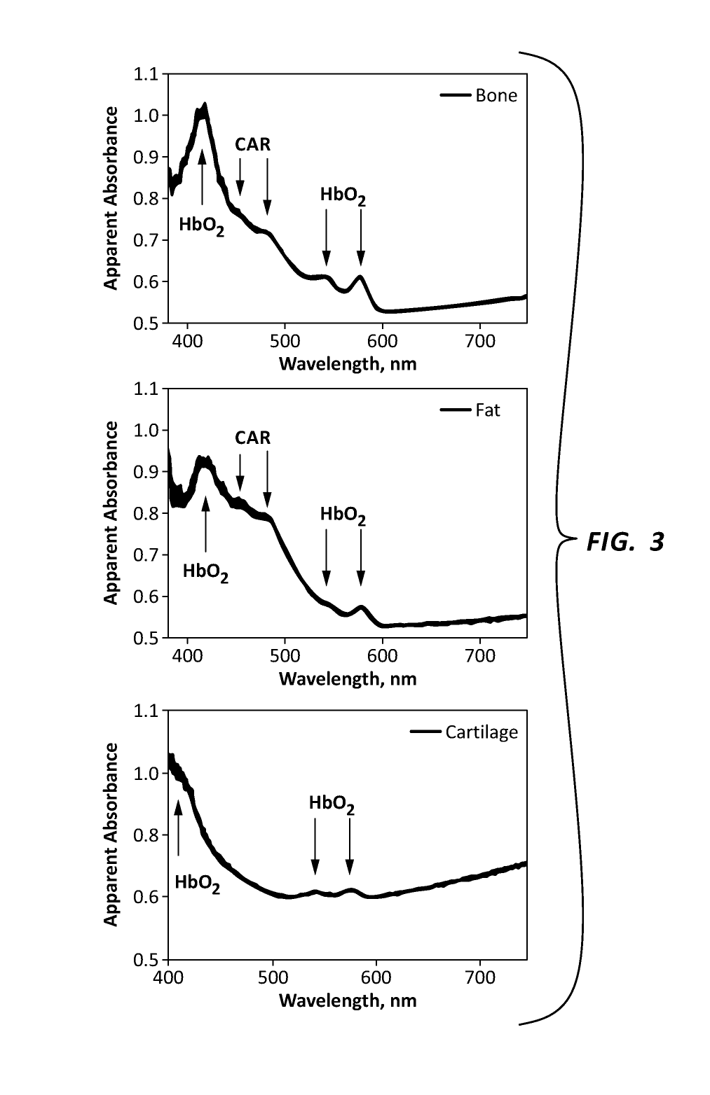

FIG. 3 illustrates graphs of the apparent absorbance of bone, fat, and cartilage respectively for wavelengths between ˜400 and 750 nm (the visible spectrum) obtained using the apparatus described above. Each chart indicates various bands where the presence of hemoglobin (HbO₂) and carotenoids (CAR) in the sample increase the sample's apparent absorbance. Apparent absorbance is intended to represent a quantification of the amount of light that is reflected by a sample (i.e. the intensity). Generally, the apparent absorption is inversely proportional to the measured reflectivity, i.e. if an absorber like carotenoids is present in a specific wavelength region, the reflectivity in the same region is attenuated accordingly. In this sense, apparent absorbance and reflectivity may be used synonymously.

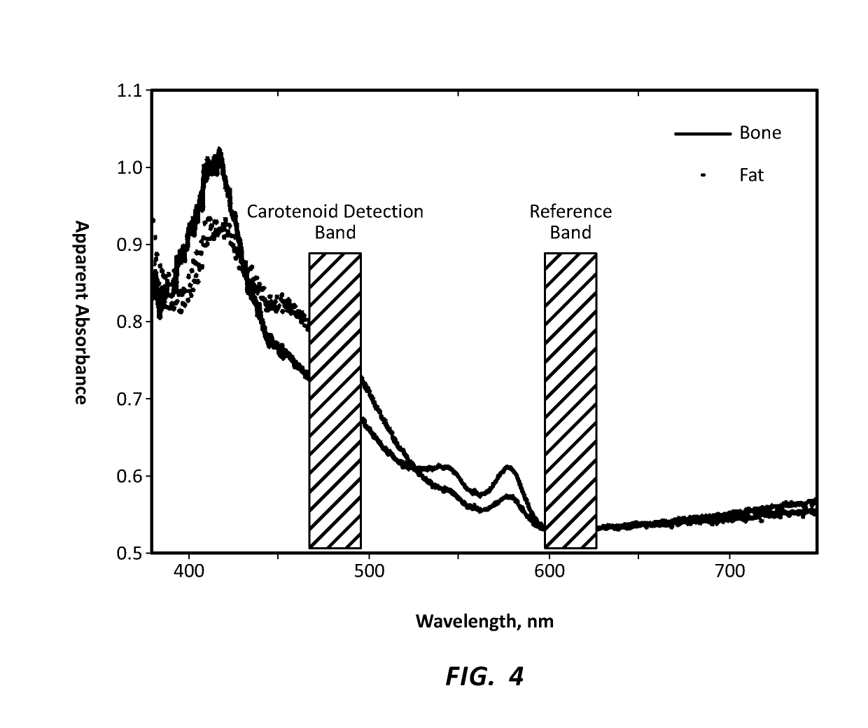

Each graph indicates a high apparent absorbance of wavelengths in the 420 nm range due to the presence of hemoglobin in bone, fat, and cartilage. Each graph also indicates a relative increase in the apparent absorbance of wavelengths in the 550-600 nm range that is also due to the presence of hemoglobin. Further, the bone and fat graphs indicate a high apparent absorbance of wavelengths in the 450-480 nm range (i.e. blue light) that is due to the presence of carotenoids. Finally, each graph indicates generally low constant values in the apparent absorbance of wavelengths of ˜600-620 nm (i.e. red light, meaning that neither hemoglobin nor carotenoids absord a significant amount of red light). These last two ranges are shown in FIG. 4 as the carotenoid detection band and the reference band respectively.

Accordingly, a greater apparent absorbance of blue light indicates a relatively higher concentration of carotenoids in the sample. However, the detected apparent absorbance of blue light only provides a relative amount that can vary based on the sample. Therefore, to determine a quantified value for carotenoid concentration in a sample, the sample's apparent absorbance of blue light is compared to the sample's apparent absorbance of red light (because the apparent absorbance of red light is generally unaffected by the presence of carotenoids in the sample). In other words, the apparent absorbance of red light acts as a baseline.

As such, the apparent absorbance and the directly correlated concentration of carotenoids in a sample can be quantified using the formula:

log(AA_{Blue-}AA_{Red})

where AA_{Blue} is the sample's apparent absorbance of blue light, AA_{Red} is the sample's apparent absorbance of red light, and log is the logarithm to base 10. Accordingly, the logarithm of a larger difference between AA_{Blue} and AA_{Red} indicates a higher concentration of carotenoids in the sample.

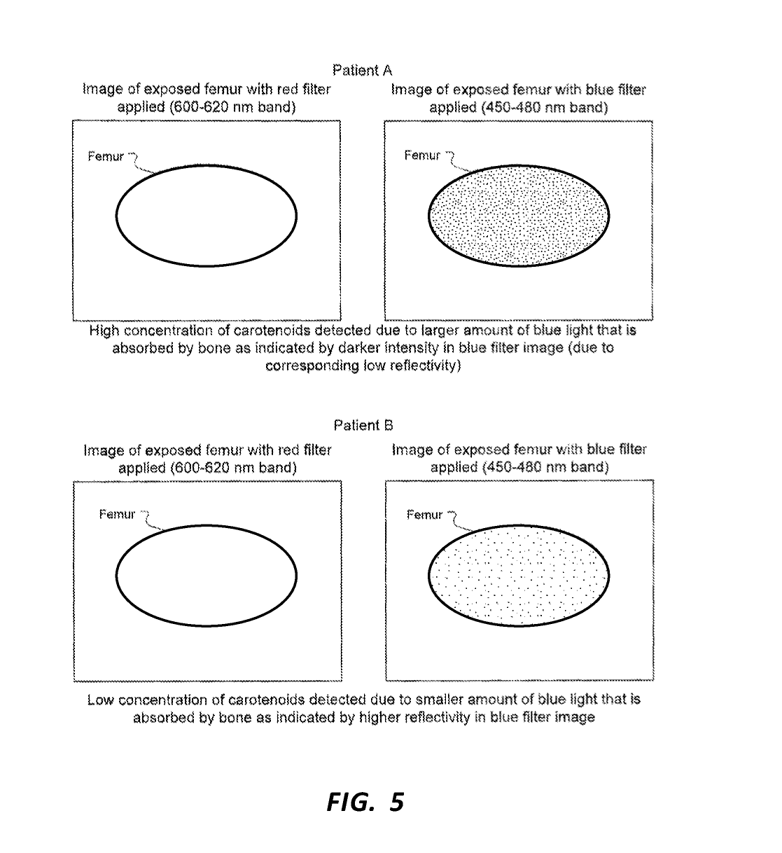

FIG. 5 provides a simplified representation of how this comparison can be performed. FIG. 5 includes two sets of theoretical images obtained by detecting the intensity of light reflected from the exposed femur of two patients.

Patient A's red filter image is only slightly gray, indicating that a very high percentage of the red light was reflected by the femur. In contrast, Patient A's blue filter image is very dark indicating that a substantial percentage of the blue light was absorbed by the femur (i.e. by the carotenoids contained in the femur), and a corresponding low percentage of the light was reflected toward the detector.

In contrast, Patient B's red filter image is again only slightly gray indicating that a high percentage of the red light was again reflected by the femur.

However, Patient B's blue filter image is only slightly darker than the red light image, indicating that a substantial percentage of the blue light was reflected by the femur in this case (i.e. because a relatively low concentration of carotenoids is present in Patient B's femur).

From these sets of images, it can be determined that Patient A's femur contains a larger concentration of carotenoids than Patient B's femur because the difference between the intensities in the blue and red filter images is greater for Patient A than for Patient B. A quantified concentration for each patient can be estimated using the equation provided above.

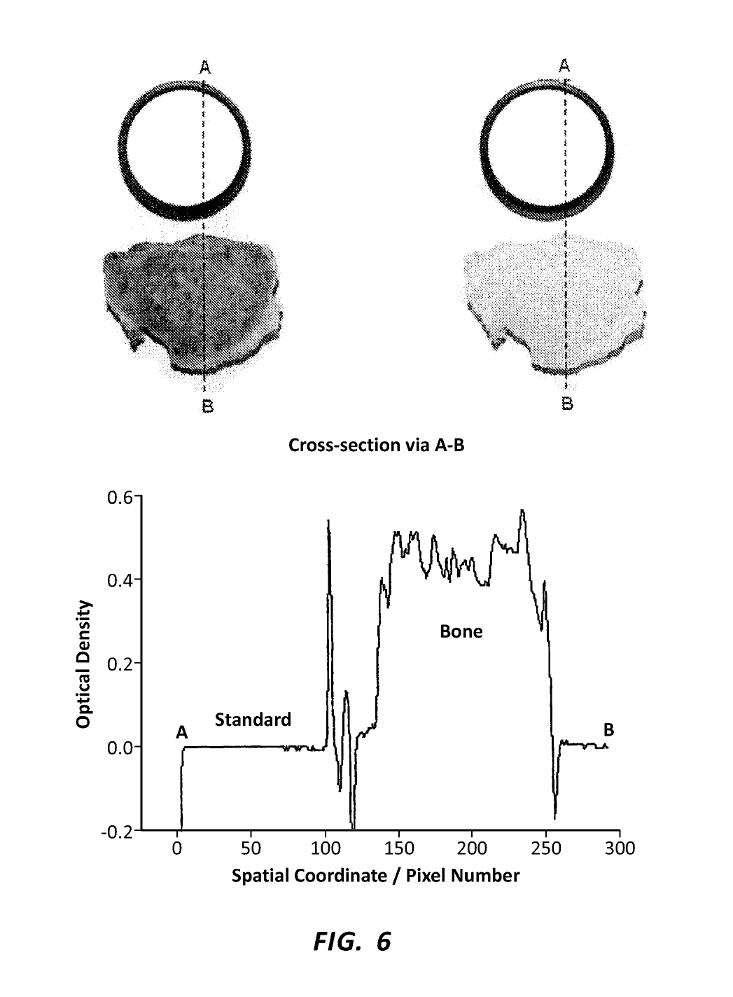

It is noted that the example of FIG. 5 is overly simplified for sake of illustration. An actual image would vary in intensity due to various factors such as the different concentrations of carotenoids in different areas of the bone or in different tissues as well as the differences in the wavelengths that each carotenoid absorbs. This is shown as an example in FIG. 6, where the imaging apparatus of FIG. 2 was used to image and measure the local concentrations of carotenoids in an excised bone sample. The upper left panel shows images of a white standard and spongy bone sample obtained, respectively, when selectively recording/filtering out only the “Carotenoid Detection Band”, i.e. the ˜480-500 nm spectral region. The upper right panel shows the corresponding images for the same white standard and bone sample when selectively recording/filtering out only the “Reference Band”, i.e. the ˜600-620 nm spectral region. The lower panel shows a spatially resolved plot of the apparent optical density of the bone carotenoid levels along the direction A-B. This was achieved by taking the common logarithm of the difference of the two corresponding images and displaying only the data for points along the direction A-B. The white standard yields zero carotenoid content, as it should; the spongy bone sample exhibits carotenoid levels as high as 0.5 OD on average over most of its central surface.

The above described apparatus employs a single light source emitting light over the entire visible light spectrum. However, the present invention can also be implemented using lights that only emit wavelengths in the blue and red bands. In other words, different configurations of apparatus 100 can be used as long as the blue and red wavelengths can be isolated from reflected light. Apparatus 100 may be preferred in most situations because of its simplicity.

Similarly, in some embodiments, multispectral imaging module 102 can be a component of camera 103 (e.g. of a camera lens or of internal circuitry of the camera, or a single camera with two dedicated light sensitive pixel arrays for red and blue, respectively, or a camera with a multi-color pixel array, that can be read out separately for red and blue light). In other words, a specialized camera capable of separately detecting the intensity of reflected red and blue wavelengths can be used in the same manner described above to quantify the concentration of carotenoids in a sample. The light source 101 could also be incorporated into such cameras. Again, apparatus 100 may generally be preferred in most situations due to its simplicity.

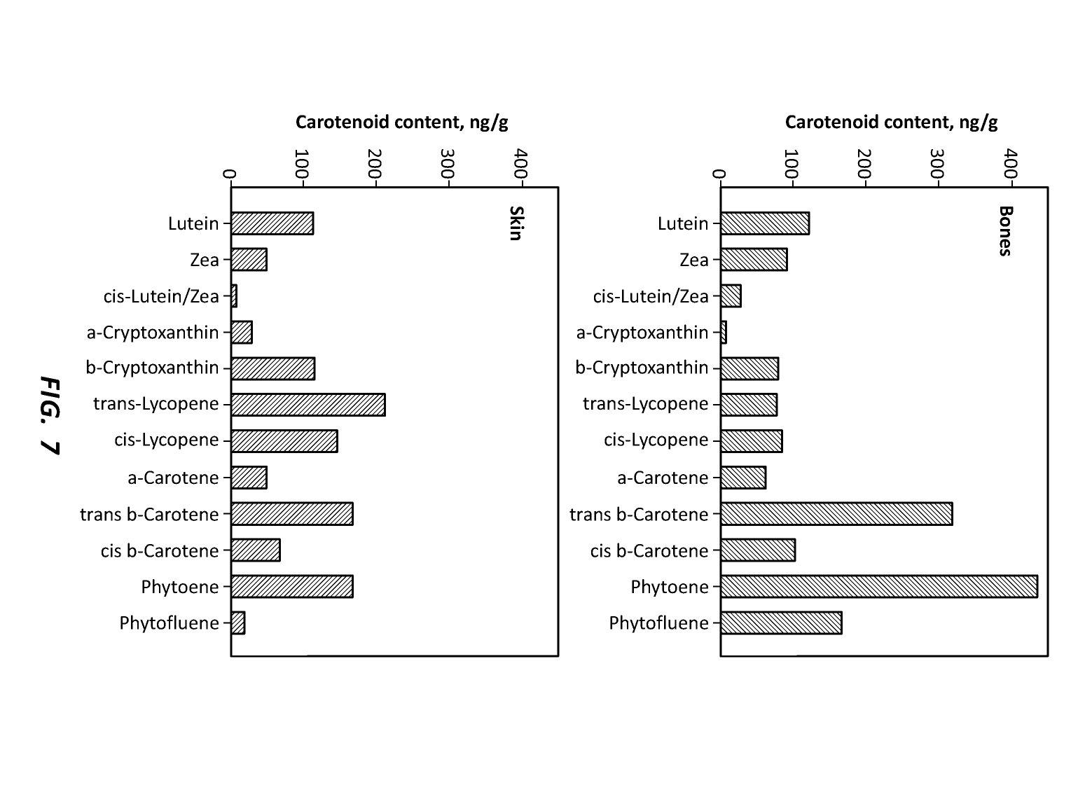

In addition to quantifying the carotenoid concentration in a patient's exposed bone or other surrounding tissue, the present invention can also be used to enable the estimation of bone carotenoid concentrations and other surrounding tissues using existing skin carotenoid tests such as those described in the background. The reason for this possibility is that the assortment of carotenoid compounds in bone and surrounding fat tissue is similar to the assortment of carotenoid compounds in skin, as illustrated in FIG. 7, which shows histograms for HPLC-derived carotenoid concentrations in 9 excised femoral bone and 8 skin samples, respectively. Due to these findings, a high correlation may exist between skin levels on one hand and bone and surrounding tissue carotenoid levels. As bone and surrounding tissue carotenoid concentrations are determined using the apparatus and method of the present invention, a correlation table can be generated.

For example, when the carotenoid concentration of a patient's exposed bone is determined, a skin test can also be performed, and a correlation between the two can be recorded. As sufficient tests are performed, the correlation table can be relied upon to estimate a patient's bone carotenoid concentration using non-invasive skin tests. In other words, if a skin test performed on a patient provides a skin carotenoid concentration of X, the correlation table can be accessed to determine the common bone carotenoid concentration when a patient's skin concentration is X.

The correlation table can be particularly useful when a patient's carotenoid concentrations are tracked over a duration of time. The concentration of carotenoids in skin increases or decreases more quickly than in bone. Accordingly, it can generally be assumed that an increase or decrease in skin carotenoid concentrations will lead to an increase or decrease in bone carotenoid concentrations. Because the present invention enables a correlation table to be created, such assumptions can be made to thereby eliminate the necessity of performing invasive tests to determine bone carotenoid concentrations.

Measuring or estimating the carotenoid levels of a patient's bones or surrounding tissues provides many potential benefits. For example, when a patient's bone carotenoid content can be known or estimated, the patient and doctor can be better guided in selecting an appropriate bone integration implant device (e.g. certain implant devices may be more appropriate when bone carotenoid levels are low). Similarly, if a patient's bone carotenoid content is known or estimated to be low, the patient can be encouraged to make dietary changes (e.g. consuming carotenoid-rich foods and/or carotenoid nutraceuticals), or to select medical regimens for improving bone health (e.g. taking prescription drugs or commencing an exercise regimen targeting bone health). Further, a known or estimated bone carotenoid level can be used to predict the health of other organs or systems (e.g. cardiovascular health, neurologic health, mental health, eye health, insulin sensitivity, etc.).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. An apparatus for optically detecting carotenoid concentrations in a trabecular human bone while the trabecular bone is exposed, the apparatus comprising:
a light source for emitting light onto an exposed trabecular human bone;
a module for receiving a portion of the light that is reflected from the bone;
a camera for capturing an image of a first band of the reflected light and an image of a second band of the reflected light; and
a computer system that compares the images to determine a difference between the intensities in the first and the second bands, and generates a value, based on the difference, that represents a carotenoid concentration in the exposed human bone.

2. The apparatus of claim 1, wherein one or more of the light source, the module, or the computer system is part of the camera.

3. The apparatus of claim 1, wherein the first band encompasses wavelengths between 600 and 620 nm.

4. The apparatus of claim 1, wherein the second band encompasses wavelengths between 450 and 480 nm.

5. The apparatus of claim 1, wherein the intensities of the first and second bands represent an apparent absorption of wavelengths in the first and second bands by carotenoids contained in the bone.

6. The apparatus of claim 5, wherein the value is generated by taking the common logarithm of the difference between the apparent absorption in the second band and the apparent absorption in the first band.

7. The apparatus of claim 6, wherein a higher value indicates a higher concentration of carotenoids in the bone.

8. The apparatus of claim 1, wherein the computer system is further configured to receive measured concentrations of carotenoids in human skin of a first patient and correlate the measured concentrations in the skin with measured concentrations in bone of the first patient.

9. The apparatus of claim 8, wherein the computer system is further configured to:
receive a measured concentration of carotenoids in human skin of a second patient;
determine that the measured concentration in the skin of the second patient is substantially the same as the measured concentration in the skin of the first patient;
identify the measured concentration in the bone of the first patient that is correlated with the measured concentration in the skin of the first patient; and
output the measured concentration in the bone of the first patient as an estimated concentration in the bone of the second patient.

10. The apparatus of claim 1, wherein the emitted light is white light and the first band comprises wavelengths in the red range and the second band comprises wavelengths in the blue range.

11. The apparatus of claim 10, wherein the module further comprises a splitter that splits the reflected white light, and a first and a second bandpass filter such that a portion of the reflected white light passes through the first band pass filter, and a portion of the reflected white light passes through the second band pass filter.

12. The apparatus of claim 12, wherein the module further comprises a plurality of mirrors for routing each portion of the reflected white light to the camera.

13. The apparatus of claim 1, wherein the module comprises a portion of a lens of the camera.

14. A method for optically detecting carotenoid concentrations in a trabecular human bone while the bone is exposed, the method comprising:
receiving light that is reflected from an exposed trabecular human bone of a first patient;
filtering a first band of the reflected white light;
filtering a second band of the reflected white light;
capturing an image of each of the first and the second bands;
determining a difference between the intensities in the first and the second bands; and
outputting a value based on the determined difference, the value representing a carotenoid concentration in the exposed human bone.

15. The method of claim 14, further comprising:
storing the value representing the carotenoid concentration in the bone with a corresponding value representing the carotenoid concentration in skin of the first patient.

16. The method of claim 15, further comprising:
receiving a second value representing a carotenoid concentration in human skin of a second patient;
determining that the second value is substantially the same as the stored value representing the carotenoid concentration in the skin of the first patient; and
outputting an estimated value of the carotenoid concentration of bone of the second patient, the estimated value comprising the stored value representing the carotenoid concentration in the bone of the first patient.

17. The method of claim 14, wherein the first band encompasses wavelengths between 600 and 620 nm.

18. The method of claim 14, wherein the second band encompasses wavelengths between 450 and 480 nm.

19. The method of claim 14, wherein determining a difference between the intensities in the first and the second bands comprises comparing an apparent absorption of wavelengths in the first and second bands by carotenoids contained in the bone.

20. An apparatus for optically detecting carotenoid concentrations in a trabecular human bone and surrounding tissue while the trabecular bone and surrounding tissue are exposed, the apparatus comprising:
a light source for emitting light onto an exposed trabecular human bone and surrounding tissue;
a module for receiving a portion of the light that is reflected from the bone and surrounding tissue;
a camera for capturing an image of a first band of the reflected light and an image of a second band of the reflected light; and
a computer system that compares the images to determine a difference between the intensities in the first and the second bands, and generates a value, based on the difference, that represents a carotenoid concentration in the exposed human bone and surrounding tissue.
